(19) United States
(12) Patent Application Publication
(10) Pub. No.: US 2021/0220295 A1
(43) Pub. Date: Jul. 22, 2021

(54) TREATMENT OF CANCER BY GUANIDINIUM DERIVATIVES

(71) Applicant: UCAR Health GmbH, Munich (DE)

(72) Inventor: Dilek UCAR, Cesme/Izmir (TR)

(21) Appl. No.: 17/045,392

(22) PCT Filed: Jun. 4, 2018
(86) PCT No.: PCT/TR2018/050150

§ 371 Date: Oct. 5, 2020

(51) Int. Cl. A61K 31/155; A61K 38/51; A61K 45/06; A61K 47/60; A61K 38/48

(52) U.S. Cl. CPC A61K 31/155; A61K 38/51; A61K 38/4813; A61K 47/60; A61K 45/06

(57) ABSTRACT

The present invention relates to a combination therapy for the treatment of cancer, particularly to combinations of oligo(2-(2-ethoxy)ethoxy ethyl guanidinium chloride), poly(hexamethylendiamine guanidiniumchloride), polyetheramines, triethyleneglycol diamine, enzymes, PGPR, amino acids, antioxidants like humic acids and some natural products like phytotherapeutic plant extracts. The combination therapy of the present invention shows enhanced anti-cancerous therapeutic effects compared to the effect of each of the components administered alone. In some embodiments, the combination therapy provide for a synergistic anti-cancer effect. A liposomal drug composition comprising; A dimeric or polymeric guanidine derivative or polyetheramines, triethyleneglycol diamine, enzymes, PGPR, amino acids, antioxidants like humic acids and some natural products like phytotherapeutic plant extracts a pharmaceutically acceptable salt thereof as drug substance, and a lipid modified by polyethylene glycole (PEG).

### FIELD OF THE INVENTION

This invention relates to the field of cancer therapy. The present invention relates to a combination therapy for the treatment of cancer, particularly to combinations of oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extractsand at least one additional anti-cancer therapeutic agent, wherein the combination therapy shows enhanced anti-cancer effect.

### BACKGROUND OF THE INVENTION

Cancer is a class of diseases in which a group of cells display uncontrolled growth, invasion and sometimes metastasis. Cancer affects people at all ages with the risk for most types increasing with age. Cancer causes about 13% of all human deaths.

Breast cancer is the leading cause of cancer in women and the second cause for women's mortality.

Only 5-10% of the most abundantly occurring human breast cancers are familial breast cancers, induced by deficiencies and mutations of the tumor suppressor genes brca1 and brca2.

All other human breast cancers are not induced by mutations of the tumor suppressor genes brca1 and brca2.

Tentori et al., Pharmacological research 52: 25-33 (2005) and Graziani et al. Pharmacological research 52: 109-118 (2005) review the use of several poly (ADP-ribose) polymerase (PARP) inhibitors (also named poly (ADP-ribose) synthetases and poly (ADP-ribose) transferases) in contributing to the treatment of cancer in combination with cytotoxic drugs.

Bryant et al., Nature 434, 913-917 (2005) and Farmer et al., Nature 434, 917-921 (2005) demonstrate that certain PARP inhibitors (such as AG14361) kill brca1 and brca2 deficient malignant cancer cells without affecting wild-type MCF-7 breast cancer cells. According to Bryant et al., supra, the sensitivity to the PARP inhibitor appears to be a direct consequence of the brca2 defect. Bryant et al., supra, further show that the survival of MCF7 cancer cells was reduced with PARP inhibitors only when brca2 was depleted from these cells.

In addition, De Soto et al., Int. J. Med. Sci, 3, 117-123 (2006) reviewed several papers showing, apart from the findings in Bryant et al., supra, and Farmer et al., supra, that CAPANI cells (which are deficient in brca2) were not inhibited by certain PARP inhibitors (such as NU1025), but were inhibited by other PARP inhibitors (such as KU0058684). Also, Bryant et al., supra, showed that only 50% MCF-7 brca1+/+cells were eradicated by exposure for 10 consecutive days to the potent PARP inhibitor AG14361 (10 μM).

Pellicciari et al., (2003), Farmaco 58, 851 and Chiarugi et al. (2003), J. Pharmacol. Exp. Ther. 305, 943 describe the PARP-1 inhibitor Tiq-A (4H-thieno[2,3-c]isoquinolin-5-one) and its potential as neuroprotective agent.

M. Banasik, et al., J. Biol. Chem. 267, 1569 (1992) describe the PARP inhibitor Phen (6(5H)-phenanthridinone). D. Weltin, et al., Int. J. Immunopharmacol. 17, 265 (1995) describe immunological properties of Phen; D. Weltin, et al., Int. J. Radiat. Biol. 72, 685 (1997) describe the ability of Phen to increase radiation induced inhibition of cell proliferation. M. R. Cookson, et al, J. Neurochem. 70, 501 (1998) describe that Phen prevented cell death induced by hydrogen peroxide or peroxynitrite. D. S. Richardson, et al.; Adv. Exp. Med. Biol. 457, 267 (1999) describe that pretreatment with Phen and 3-aminobenzamide (3AB) in HL-60 myeloid leukemia cell lines resulted in resistance to apoptotic death rather than potentiation thereof.

F. Bernges & W. J. Zeller, J. Cancer Res. Clin. Oncol. 122, 665 (1996) describe that the PARP inhibitor 3-AB had no effect on the cytotoxicity of cisplatin.

WO 01/42219 discloses the PARP inhibitor PJ-34 (N-(6-oxo-5,6-dihydro-phenanthridin-2-yl)-N,N-dimethylacetamide, HC1) as a compound protecting against neuronal cell death induced by stroke or inflammation.

Tentori et al., supra, describes PJ-34 and its protective effects against cardiac dysfunction.

Pacher et al., (2002) J. Am. Coll. Cardiol. 40, 1006-1009 injected PJ-34 in rodents for a 10 week period to diminish cardiomyocytes cell death after cardiac stroke and to avoid chronic heart disease.

Cohen-Armon M. et al., (2007) Mol Cell 25, 297-308; Homburg et al., (2000) J. Cell Biol. 150:293-308; Visochek et al., (2005) J. Neurosci. 25:7420-742 describe that the survival of non-dividing cells, such as brain cortical neurons or cardiomyocytes is not affected following treatment with PJ-34.

Abdelkarim et al., (2001) Int. J. Mol. Med, 7, 255-260 and Park et al., (2004) Stroke, 35, 2896-2901 describe the neuroprotective effect of PJ-34 after stroke both in vivo and in vitro.

Cancer or neoplasm is a malignant growth characterized by unregulated proliferation of cells. Cancerous cells propagate from a single cell and can be multiplied to develop into tumor tissues. The cancerous cells can invade nearby tissues and spread through the bloodstream and lymphatic system to other parts of the body (metastasis). Most cancers can be treated, and some cured, depending on the specific type, location, and stage of development. Once diagnosed, cancer is usually treated with one or a combination of surgery, chemotherapy and radiotherapy.

Surgery generally is only effective for treating the earlier stages of cancer and in removing tumors located at certain sites, for example, in the breast, colon, and skin. However, it cannot be used in the treatment of tumors located in other areas inaccessible to surgeons, nor in the treatment of disseminated neoplastic conditions such as leukemia.

Radiation therapy is only effective for treating clinically localized disease at early and middle stages of cancer, and is not effective for the late stages of cancer with metastasis. Radiation is generally applied to a defined area of the subject's body which contains abnormal proliferative tissue, in order to maximize the dose absorbed by the abnormal tissue and minimize the dose absorbed by the nearby normal tissue. However, it is difficult (if not impossible) to selectively administer therapeutic radiation to the abnormal tissue. Thus, normal tissue proximate to the abnormal tissue is also exposed to potentially damaging doses of radiation throughout the course of treatment.

The majority of chemotherapeutic drugs can be divided into: alkylating agents (e.g. cyclophosphamide), antimetabolites (e.g. fluorouracil), plant alkaloids (e.g. paclitaxel), topoisomerase inhibitors (e.g. topotecan), and cytotoxic antibiotics (e.g. daunorubicin). All of these drugs impair cell division or DNA synthesis and functions. However, most of the chemotherapeutic drugs cause undesirable systemic effects such as cardiac or renal toxicity, marrow aplasia, alopecia, nausea and vomiting.

Immunotherapy is now an emerging treatment modality for a variety of cancers and several promising treatments have been already approved and are being tested in clinical trials. Antibodies are useful in cancer therapy because they can recognize tumor-associated antigens expressed at higher density on malignant compared to normal cells Immunotherapy can be used as a single therapy or in combination with traditional drug therapies. In the past two decades antibodies have been the fastest growing class of pharmaceutical proteins.

The shortcomings of the presently available cancer therapies have lead to the search for combinations treatments that may answer at least some of these shortcomings. For example, U.S. Patent Application Publication No. 2003/0108623 discloses pharmaceutical compositions containing plant essential oil compounds including oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extractsor mixtures or derivatives thereof, with one or more signal transduction modulators, for the prevention and treatment of cancer. The pharmaceutical composition can be administered with a conventional cancer treatment, e.g., tamoxifen.

U.S. Patent Application Publication No. 2004/0092583 discloses the use of incensole and/or furanogermacrens, derivatives, metabolites and precursors thereof in the treatment of neoplasia, particularly resistant neoplasia and immunodysregulatory disorders. The compounds, oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts being one among many others, can be administered alone or in combination with conventional chemotherapeutic, anti-rival or anti-parasite agents, and further in combination with radiation and/or surgery.

U.S. Patent Application Publication No. 2008/0113042 discloses pharmaceutical compositions and methods for cancer treatment based on combinational use of conventional anticancer agents and geranium oil or compounds thereof. The compositions are disclosed to be effective in broad range of cancer types.

Due to the severity and breadth of cancer diseases, there is a recognized need for additional effective means and methods for treating cancer with improved outcome.

### SUMMARY OF THE INVENTION

The present invention provides a combination therapy for treating various types of cancer. Particularly, the present invention provides compositions and methods combining oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts that show significant enhancement of the anti-cancer effect, which is preferably synergistic.

The present invention is based in part on the unexpected discovery that oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts show a synergistic inhibiting effect on the proliferation of various cancerous cell types. This phenomenon was observed in a wide range of cancer cell lines representing different types of cancer, and further in an in vivo model of colorectal cancer.

Without wishing to be bound by any particular theory or mechanism of action this synergistic effect may be attributed to the capability of oligo(2-(2-ethoxy)ethoxy ethyl guanidinium chloride), poly(hexamethylendiamine guanidiniumchloride), polyetheramines, Triethyleneglycol diamine, polyetheramines, Triethyleneglycol diamine, enzymes, PGPR, amino acids, antioxidants like humic acids and some natural products like phytotherapeutic plant extracts to significantly enhance the effect of known anti-cancer agents, including chemotherapeutic drugs as well as biologic drugs, particularly antibodies. Such combinations may therefore be used for treating wide range of cancers.

The combination therapy is particularly advantageous, since not only the anti-cancerous effect is enhanced compared to the effect of each compound alone, the dosage of each agent in a combination therapy can be reduced as compared to monotherapy with each agent, while still achieving an overall anti-tumor effect. In addition, due to the synergistic effect, the total amount of drugs administered to a patient can advantageously be reduced, which may result in decreased side effects.

Thus, according to one aspect, the present invention provides a method for treating cancer, the method comprising administering to a subject in need thereof (a) an effective amount of oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts and (b) an effective amount of at least one additional anti-cancer agent to provide a combination therapy having an enhanced therapeutic effect compared to the effect of the oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts and the at least one additional anti-canceragent each administered alone. According to certain exemplary embodiments, the combination therapy has a synergistic therapeutic effect. According to this embodiment, the combination therapy produces a significantly better anti-cancer result (e.g., cell growth arrest, apoptosis, induction of differentiation, cell death, etc.) than the additive effects achieved by each individual constituent when administered alone at a therapeutic dose.

According to certain embodiments, the cancer is a solid tumor. According to other embodiments, the cancer is a non-solid tumor.

According to some embodiments, the solid-tumor cancer is selected from the group consisting of tumors of the central nervous system, breast cancer, prostate cancer, skin cancer (including basal cell carcinoma, cell carcinoma, squamous cell carcinoma and melanoma), cervical cancer, uterine cancer, lung cancer, ovarian cancer, testicular cancer, thyroid cancer, astrocytoma, glioma, pancreatic cancer, mesotheliomas, gastric cancer, liver cancer, colon cancer, rectal cancer, renal cancer including nephroblastoma, bladder cancer, oesophageal cancer, cancer of the larynx, cancer of the parotid, cancer of the biliary tract, endometrial cancer, adenocarcinomas, small cell carcinomas, neuroblastomas, adrenocortical carcinomas, epithelial carcinomas, desmoid tumors, desmoplastic small round cell tumors, endocrine tumors, Ewing sarcoma family tumors, germ cell tumors, hepatoblastomas, hepatocellular carcinomas, non-rhabdomyosarcome soft tissue sarcomas, osteosarcomas, peripheral primitive neuroectodermal tumors, retinoblastomas and rhabdomyosarcomas. Each possibility represents a separate embodiment of the present invention.

According to other embodiments, the non-solid tumor is a blood cancer, including, for example, leukemia and lymphoma. Each possibility represents a separate embodiment of the present invention.

According to certain embodiments, the at least one additional agent is a biologic drug, particularly an antibody. According to some embodiments, the antibody is selected from the group consisting of Acinetobacter, Achromobacter, Aereobacter, Agrobacterium, Alcaligenes, Artrobacter, Azospirillum, Serratia, Bacillus, Burkholderia, Enterobacter, Erwinia, Flavobacterium, Microccocus, Pseudomonas, Rhizobium ve Xanthomonas.

According to certain embodiments, the additional anti-agent cancer is known to be effective in treating a particular type of cancer.

The terms “combination therapy” or “combined treatment” or “in combination” as used herein denotes any form of concurrent or parallel treatment with at least two distinct therapeutic agents.

According to certain embodiments, the oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts and the at least one additional anti-cancer agent are administered simultaneously, either in the same composition or in separate compositions. According to other embodiments, the oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts and the at least one additional anti-canceragent are administered sequentially, i.e., the oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts are administered either prior to or after the administration of the additional anti-cancer agent. In some embodiments, the administration of the oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts and the additional anti-cancer agent are concurrent, i.e., the administration period of the oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extractsand that of the agent overlap with each other. In some embodiments, the administration of the oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extractsand the additional anti-cancer agent are non-concurrent. For example, in some embodiments, the administration of the oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extractsis terminated before the additional agent is administered. In some embodiments, the administration of the additional anti-cancer agent is terminated before the oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts are administered.

According to certain typical embodiments, the oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts and the additional anti-cancer agent are administered within a single therapeutic composition. According to some embodiments, the therapeutic composition further comprises therapeutically acceptable diluents or carrier.

According to certain embodiments, the oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts are administered in an amount of from 0.1 mg/Kg body weight to 100 mg/Kg body weight. According to other embodiments, the oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts are administered at an amount of from 0.5 mg/Kg body weight to 20 mg/Kg body weight. According to additional embodiments, the oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts are administered at an amount of from 1.0 mg/Kg body weight to 10 mg/Kg body weight.

According to certain embodiments, the at least one additional anti-cancer agent is administered at the therapeutic amount known to be used for treating the specific type of cancer. According to other embodiments, the at least one additional anti-cancer agent is administered in an amount lower than the therapeutic amount known to be used for treating the disease.

The present invention also contemplates a method for inhibiting cancer cell proliferation, comprising contacting cancer cells with oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts in combination with at least one additional anti-cancer agent, wherein the combination provides an enhanced anti-cancerous effect compared to the effect of the oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts and the at least one additional anti-cancer agent each administered alone. According to certain exemplary embodiments, the combination of oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts and the at least one additional anti-cancer agent has a synergistic effect.

In yet other embodiments the present invention relates to the use of oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts in combination with at least one other anti-cancer agent, wherein the oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts and the at least one other anti-cancer agent together provide an enhanced therapeutic effect, preferably a synergistic therapeutic effect.

According to an additional aspect, the presented invention provides the use of an effective amount of oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts for the preparation of a medicament for treating cancer to be administered in combination with at least one additional anti-cancer agent, thereby enhancing the anti-cancerous effect compared to the effect of each of the medicament comprising the oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extractsand the at least one additional anti-cancer agent. According to certain embodiment, the anti-cancerous effect is synergistic.

According to certain exemplary embodiments, the medicament consists of the oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts as the sole active agent.

According to certain embodiments, the medicament comprising or consisting of the oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts are to be administered simultaneously with the at least one additional anti-cancer agent. According to other embodiments, the medicament comprising or consisting of the oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts and the at least one additional anti-cancer agent are to be administered sequentially. In some embodiments, the medicament comprising or consisting of the oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts and the at least one additional anti-cancer agent are to be administered concurrently. In yet other embodiments, the medicament comprising or consisting of the oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts and the at least one additional anti-cancer agent are to be administered non-concurrently. According to yet additional embodiments, the oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts and the at least one additional anti-cancer agent are to be administered in the same medicament.

According to yet additional aspect, the present invention provides a composition for treating cancer, the composition comprising a first component consisting of an effective amount of oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts and a second component comprising an effective amount of at least one additional anti-cancer agent. The collective amount of oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts and the at least one additional anti-cancer agent provides for an enhanced therapeutic anti-cancer effect. According to certain embodiments, the collective amount of oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts and the at least one additional anti-cancer agent provides for a synergistic therapeutic anti-cancer effect. According to certain exemplary embodiments, the composition further comprises a pharmaceutically acceptable diluents or carrier.

According to certain embodiments, the composition comprises oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts at a concentration range of from about 0.01% to about 99% (v/v) relative to the total volume of the composition. According to certain exemplary embodiments, the concentration of oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts are from about 0.1% to 80% or from 0.1% to 70% (v/v) relative to the total volume of the composition.

According to certain embodiments, the composition is administered in an amount as to provide oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts at an amount of from 0.1 mg/Kg body weight to 100 mg/Kg body weight. According to certain exemplary embodiments, the composition is administered in an amount as to provide oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts at an amount of from 0.5 mg/Kg body weight to 20 mg/Kg body or from 1.0 mg/Kg body weight to 10 mg/Kg body weight.

According to some aspects, the present invention provides the use of an effective amount of oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts and an effective amount of at least one additional anti-cancer agent for the preparation of a medicament for treating cancer, wherein the collective amount of oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts and the at least one additional anti-cancer agent provides for an enhanced therapeutic anti-cancer effect. According to certain embodiments, the collective amount of oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts and the at least one additional anti-cancer agent provides for a synergistic therapeutic anti-cancer effect.

### DETAILED DESCRIPTION OF THE INVENTION

As used herein, the term “cancer” includes all cancers and cancer metastases, including sarcomas, carcinomas and other solid and non-solid tumor cancers. Solids cancer include but are not limited to tumors of the central nervous system, breast cancer, prostate cancer, skin cancer (including basal cell carcinoma cell carcinoma, squamous cell carcinoma and melanoma), cervical cancer, uterine cancer, lung cancer, ovarian cancer, testicular cancer, thyroid cancer, astrocytoma, glioma, pancreatic cancer, stomach cancer, liver cancer, colon cancer, renal cancer, bladder cancer, oesophageal cancer, cancer of the larynx, cancer of the parotid, cancer of the biliary tract, rectal cancer, endometrial cancer, adenocarcinomas, small cell carcinomas, neuroblastomas, mesotheliomas, adrenocortical carcinomas, epithelial carcinomas, desmoid tumors, desmoplastic small round cell tumors, endocrine tumors, Ewing sarcoma family tumors, germ cell tumors, hepatoblastomas, hepatocellular carcinomas, non-rhabdomyosarcome soft tissue sarcomas, osteosarcomas, peripheral primitive neuroectodermal tumors, retinoblastomas, rhabdomyosarcomas, Wilms tumors, and the like. According to certain embodiments of the present invention, the cancer is selected from the group consisting of cancers of the gastrointestinal tract, pancreatic cancer and prostate cancer. Each possibility represents a separate embodiment of the present invention. According to some embodiments, the cancer of the gastrointestinal tract is selected from the group consisting of colorectal cancer and gastric cancer. According to certain embodiments, the term “cancer” further comprises pre-cancerous lesions.

The term “subject” as used herein refers to any mammal having cancer which requires treatment. Typically, the mammal is human; however, it should be explicitly understood that the mammal can also be a companion animal, for example a dog or a cat.

The terms “treating”, “treatment” and the like are used herein to mean affecting a subject, tissue or cell to obtain a desired pharmacological and/or physiological effect. The effect may be therapeutic in terms of a partial or complete cure of the cancer. “Treating” as used herein covers any treatment of cancer in a subject; inhibiting the cancer, i.e. arresting its development; or relieving or ameliorating the effects of the cancer, i.e., cause regression of the tumor or of the effects of the cancer.

The term “anti-cancer” as used herein in reference to “anti-cancer agent”, “anti-cancer therapeutic effect” “anti-cancerous effect” and the like is meant in its broadest scope as in known in the art, and includes the activities of arrest of cell growth, induction of apoptosis, induction of differentiation, cell death and the like.

As used herein, the terms “effective amount” refers to an amount of oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts or another anti-cancer agent according to the teachings of the present invention that is effective in treating cancer as defined hereinabove. The specific “effective amount” will vary according to the particular condition being treated, the physical condition and clinical history of the subject, the duration of the treatment and the nature of the combination of agents applied and its specific formulation. As used herein, the term “therapeutically effective amount” refers to the amount of oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts and/or the at least one additional anti-cancer agent known in the art to be effective in treating cancer cells/disease of a particular type. According to certain embodiments, the “effective amount” according to the teachings of the present invention is lower compared to the “therapeutically effective amount” as is known in the art.

The term “enhanced effect” and its various grammatical variations is used herein to refer to an interaction between oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts and at least one other agent wherein the observed effect (e.g., cytotoxicity) in the presence of the drugs together is significantly higher than the effect of each individual drug (e.g., cytotoxicities) administered separately. In one embodiment, the observed combined effect of the drugs is significantly higher than each of the individual effects. In certain embodiments the term significant means that the observed p<0.05.

The term “synergistic” and its various grammatical variations is used herein to refer to an interaction between oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts and at least one other agent wherein the observed effect (e.g., cytotoxicity) in the presence of the drugs together is higher than the sum of the individual effects (e.g., cytotoxicities) of each drug administered separately. In one embodiment, the observed combined effect of the drugs is significantly higher than the sum of the individual effects. In certain embodiments the term significant means that the observed p<0.05.

According to one aspect, the present invention provides a method for treating cancer, the method comprises administering to a subject in need thereof (a) an effective amount of oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts and (b) an effective amount of at least one anti-cancer agent to provide a combination therapy having an enhanced anti-cancerous effect compared to the effect of the oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts and the at least one additional anti-cancer agent each administered alone. According to certain exemplary embodiments, the combination therapy has a synergistic therapeutic effect.

Reference to a chemotherapeutic agent herein applies to the chemotherapeutic agent or its derivatives and accordingly the invention contemplates and includes either of these embodiments (agent; agent or derivative(s)). “Derivatives” or “analogs” of a chemotherapeutic agent or other chemical moiety include, but are not limited to, compounds that are structurally similar to the chemotherapeutic agent or moiety or are in the same general chemical class as the chemotherapeutic agent or moiety. The derivative or analog of the chemotherapeutic agent or moiety retains similar chemical and/or physical property (including, for example, functionality) of the chemotherapeutic agent or moiety.

Plant derived agents include taxanes, which are semisynthetic derivatives of extracted precursors from the needles of yew plants. These drugs have a novel 14-member ring, the taxane. Unlike the vinca alkaloids, which cause microtubular disassembly, the taxanes (e.g., taxol) promote microtubular assembly and stability, therefore blocking the cell cycle in mitosis. Other plant derived agents include, but are not limited to, vincristine, vinblastine, vindesine, vinzolidine, vinorelbine, etoposide, teniposide, and docetaxel.

Biologic agents suitable for use in the present invention include, but are not limited to immuno-modulating proteins, monoclonal antibodies against tumor antigens, tumor suppressor genes, kinase inhibitors and inhibitors of growth factors and their receptors and cancer vaccines. For example, the immuno-modulating protein can be interleukin 2, interleukin 4, interleukin 12, interferon El interferon D, interferon alpha, erythropoietin, granulocyte-CSF, granulocyte, macrophage-CSF, bacillus Calmette-Guerin, levamisole, or octreotide. Agents affecting cell bioenergetics affecting cellular ATP levels and/or molecules/activities regulating these levels

Recent developments have introduced, in addition to the traditional cytotoxic and hormonal therapies, additional therapies for the treatment of cancer. For example, many forms of gene therapy are undergoing preclinical or clinical trials. In addition, approaches based on the inhibition of tumor vascularization (angiogenesis) are currently under development. The aim of this concept is to cut off the tumor from nutrition and oxygen supply provided by a newly built tumor vascular system. In addition, cancer therapy is also being attempted by the induction of terminal differentiation of the neoplastic cells. Suitable differentiation agents include hydroxamic acids, derivatives of vitamin D and retinoic acid, steroid hormones, growth factors, tumor promoters, and inhibitors of DNA or RNA synthesis. Also, histone deacetylase inhibitors are suitable chemotherapeutic agent to be used in the present invention.

According to certain embodiments, the at least one additional anti-cancer agent is known to be effective in treating the cancer type affecting the subject.

Determining the dosage and duration of treatment according to any aspect of the present invention is well within the skills of a professional in the art. The skilled Artisans are readily able to monitor patients to determine whether treatment should be started, continued, discontinued or resumed at any given time. For example, dosages of the compounds are suitably determined depending on the individual cases taking symptoms, age and sex of the subject and the like into consideration. The amount of the compound to be incorporated into the pharmaceutical composition of the invention varies with dosage route, solubility of the compound, administration route, administration scheme and the like. An effective amount for a particular patient may vary depending on factors such as the condition being treated, the overall health of the patient and the method, route and dose of administration. The clinician using parameters known in the art makes determination of the appropriate dose. Generally, the dose begins with an amount somewhat less than the optimum dose and it is increased by small increments thereafter until the desired or optimum effect is achieved. Suitable dosages can be determined by further taking into account relevant disclosure in the known art.

According to certain embodiments, the oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts are administered in an amount sufficient so as to allow reduction of the normal dose of the at least one additional anti-cancer agent required to effect the same degree of treatment by at least about any of 5%, 10%, 20%, 30%, 50%, 60%, 70%, 80%, 90%, or more. According to other embodiments, the at least one additional anti-cancer agent is administered in an amount sufficient so as to allow reduction of the normal dose of the chemotherapeutic agent required to effect the same degree of treatment by at least about any of 5%, 10%, 20%, 30%, 50%, 60%, 70%, 80%, 90%, or more.

According to certain embodiments, the oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts are administered in an amount of from 0.1 mg/Kg body weight to 100 mg/Kg body weight. According to other embodiments, the oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts are administered at an amount of from 0.5 mg/Kg body weight to 20 mg/Kg body weight. According to additional embodiments, the oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts are administered at an amount of from 1.0 mg/Kg body weight to 10 mg/Kg body weight.

However, it is to be explicitly understood that lower or higher concentrations of oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts can be used, depending on the additional anti-cancer agent(s) present in the composition, the subject to be treated (age, gender, weight etc.), the type of cancer to be treated and the stage of the disease. As exemplified hereinbelow, the combination of oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts and at least one additional anti-cancer agent significantly reduced the survival of cancer cell lines. The inhibiting activity of the combination was significantly higher as compared to the additive inhibition activity of each of the component, and thus defined as synergistic effect.

The oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts and the at least one additional anti-cancer agent or a composition comprising same (“additional anti-cancer agent”) can be administered simultaneously (i.e., simultaneous administration) and/or sequentially (i.e., sequential administration).

According to some embodiments, the oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts and the at least additional anti-cancer agent are administered simultaneously. The term “simultaneous administration,” as used herein, means that the oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts and the at least additional anti-cancer agent are administered with a time separation of no more than about 15 minute(s), such as no more than about any of 10, 5, or 1 minutes. When the drugs are administered simultaneously, the oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts and the at least additional anti-cancer agent may be contained in the same composition (e.g., a composition comprising both the oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts and the at least additional anti-cancer agent) or in separate compositions are contained in one composition and the at least additional anti-cancer agent is contained in another composition).

According to other embodiments, the oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts and the at least one additional anti-cancer agent are administered sequentially. The term “sequential administration” as used herein means that the oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts and the additional anti-cancer agent are administered with a time separation of more than about 15 minutes, such as more than about any of 20, 30, 40, 50, 60 or more minutes. Either the oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts or the additional anti-cancer agent may be administered first. The oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts and the additional anti-cancer agent are contained in separate compositions, which may be contained in the same or different packages.

According to yet additional embodiments, the administration of the oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts and the at least one additional anti-cancer agent are concurrent, i.e., the administration period of the oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts and that of the at least one additional anti-cancer agent overlap with each other. In some embodiments, the administration of the oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extractsand the at least one additional anti-cancer agent are non-concurrent. For example, in some embodiments, the administration of the oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts are terminated before the at least one additional anti-cancer agent is administered. In some embodiments, the administration of the at least one additional anti-cancer agent is terminated before the oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts are administered. The time period between these two non-concurrent administrations can range from being days apart to being weeks apart.

The dosing frequency of the oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts and the at least one additional anti-cancer agent may be adjusted over the course of the treatment, based on the judgment of the administering physician. When administered separately, the oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts and the at least one additional anti-cancer agent can be administered at different dosing frequency or intervals. For example, the oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts can be administered weekly, while the at least one additional anti-cancer agent can be administered more or less frequently. In some embodiments, sustained continuous release formulation of both components may be used. Various formulations and devices for achieving sustained release are known in the art. In addition, the oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts and the at least one additional anti-cancer agent can be administered using the same route of administration or using different routes of administration.

It is to be explicitly understood that the present invention further encompasses combinations of the various administration configurations described herein. The methods described herein employing a combination of oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts and at least one additional anti-cancer agent can be performed alone or in combination with another therapy, including surgery, radiation, chemotherapy, immunotherapy, gene therapy, and the like.

Although the components of the combination therapy of the present invention can be administered alone, it is contemplated that the components of the combination will be administered in pharmaceutical compositions further containing at least one pharmaceutically acceptable carrier or excipient. Each of the components can be administered in a separate pharmaceutical composition, or the combination can be administered in one pharmaceutical composition.

Thus, according to additional aspect, the present invention provides a composition for treating cancer, the composition comprising a first component consisting of an effective amount of oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts and a second component comprising an effective amount of at least one additional anti-cancer agent. The oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts and the at least one other anti-cancer agent together provide a therapeutic anti-cancer effect which is at least enhanced compared to the effect of each of the components administered alone, and, in one embodiment, is synergistic.

According to certain exemplary embodiments, the compositions of the present invention are pharmaceutical compositions further comprising pharmaceutically acceptable diluents, excipients or carriers.

According to certain embodiments, the pharmaceutical composition comprises oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts at a concentration range of from about 0.01% to about 99% (v/v) relative to the total volume of the composition. According to certain exemplary embodiments, the concentration of oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extractsis from about 0.1% to 90% or from 0.1% to 80% or from 0.1% to 70% (v/v) relative to the total volume of the composition.

According to certain embodiments, the pharmaceutical composition is administered in an amount as to provide oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts at an amount of from 0.01 mg/Kg body weight to 100 mg/Kg body weight. According to certain exemplary embodiments, the composition is administered in an amount as to provide oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts at an amount of from 0.5 mg/Kg body weight to 20 mg/Kg body or from 1.0 mg/Kg body weight to 10 mg/Kg body weight.

The pharmaceutical compositions of the present invention can be formulated for administration by a variety of routes. The pharmaceutical compositions may be administered orally, parenterally, by inhalation spray, topically, rectally, nasally, buccally, vaginally or via an implanted reservoir. Direct administration to solid tumor is explicitly encompassed by the present invention. The pharmaceutical compositions may contain any conventional non-toxic pharmaceutically acceptable carriers, adjuvants or vehicles. Parenteral administration of the compositions may include subcutaneous, intracutaneous, intravenous, intramuscular, intraperitoneal, intraarticular, intrasynovial, intrasternal, intrathecal, intralesional and intracranial injection or infusion techniques. Typically, administration rout would be adapted according to the type of the cancer to be treated and the formulae of the compositions of the invention.

The pharmaceutical compositions of the invention can be administered locally or systemically. By systemic administration means any mode or route of administration that results in effective amounts of the active ingredients appearing in the blood or at a site remote from the route of administration of the active ingredients.

During the preparation of the pharmaceutical compositions according to the present invention the active ingredient is usually mixed with a carrier or excipient, which may be a solid, semi-solid, or liquid material. The compositions can be in the form of tablets, pills, capsules, pellets, granules, powders, lozenges, sachets, cachets, elixirs, suspensions, dispersions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

The carriers may be any of those conventionally used and are limited only by chemical-physical considerations, such as solubility and lack of reactivity with the compound of the invention, and by the route of administration. The choice of carrier will be determined by the particular method used to administer the pharmaceutical composition. Some examples of suitable carriers include lactose, glucose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water and methylcellulose. The formulations can additionally include lubricating agents such as talc, magnesium stearate, and mineral oil; wetting agents, surfactants, emulsifying and suspending agents; preserving agents such as methyl- and propylhydroxybenzoates; sweetening agents; flavoring agents, colorants, buffering agents (e.g., acetates, citrates or phosphates), disintegrating agents, moistening agents, antibacterial agents, antioxidants (e.g., ascorbic acid or sodium bisulfate), chelating agents (e.g., ethylenediaminetetraacetic acid), and agents for the adjustment of tonicity such as sodium chloride. Other pharmaceutical carriers can be sterile liquids, such as water and oils, including those of petroleum, animal, vegetable or synthetic origin, such as peanut oil, soybean oil, mineral oil, sesame oil and the like, polyethylene glycols, glycerin, propylene glycol or other synthetic solvents. Water is a preferred carrier when the pharmaceutical composition is administered intravenously. Saline solutions and aqueous dextrose and glycerol solutions can also be employed as liquid carriers, particularly for injectable solutions.

In one embodiment, in the pharmaceutical composition the active ingredient is dissolved in any acceptable lipid carrier (e.g., fatty acids, oils to form, for example, a micelle or a liposome).

For preparing solid compositions such as tablets, the principal active ingredient(s) is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation is then subdivided into unit dosage forms of the type described above containing the desired amount of the active compounds.

Any method can be used to prepare the pharmaceutical compositions. Solid dosage forms can be prepared by wet granulation, dry granulation, direct compression and the like. The solid dosage forms of the present invention may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer, which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compositions of the present invention may be incorporated, for administration orally or by injection, include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insulation include solutions and suspensions in pharmaceutically acceptable aqueous or organic solvents, or mixtures thereof, and powders. The liquid or solid compositions may contain suitable pharmaceutically acceptable excipients as described above. Preferably the compositions are administered by the oral or nasal respiratory route for local or systemic effect.

Compositions in preferably pharmaceutically acceptable solvents may be nebulized by use of inert gases. Nebulized solutions may be breathed directly from the nebulizing device or the nebulizing device may be attached to a face masks tent, or intermittent positive pressure breathing machine. Solution, suspension, or powder compositions may be administered, preferably orally or nasally, from devices that deliver the formulation in an appropriate manner.

Another formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art.

In yet another embodiment, the composition is prepared for topical administration, e.g. as an ointment, a gel a drop or a cream. For topical administration to body surfaces using, for example, creams, gels, drops, ointments and the like, the compounds of the present invention can be prepared and applied in a physiologically acceptable diluent with or without a pharmaceutical carrier. The present invention may be used topically or transdermally to treat cancer, for example, melanoma. Adjuvants for topical or gel base forms may include, for example, sodium carboxymethylcellulose, polyacrylates, polyoxyethylene-polyoxypropylene-block polymers, polyethylene glycol and wood wax alcohols.

Alternative formulations include nasal sprays, liposomal formulations, slow-release formulations, pumps delivering the drugs into the body (including mechanical or osmotic pumps) controlled-release formulations and the like, as are known in the art.

The compositions are preferably formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

In preparing a formulation, it may be necessary to mill the active ingredient to provide the appropriate particle size prior to combining with the other ingredients. If the active compound is substantially insoluble, it ordinarily is milled to a particle size of less than 200 mesh. If the active ingredient is substantially water soluble, the particle size is normally adjusted by milling to provide a substantially uniform distribution in the formulation, e.g. about 40 mesh.

It may be desirable to administer the pharmaceutical composition of the invention locally to the area in need of treatment; this may be achieved by, for example, and not by way of limitation, local infusion during surgery, infusion to the liver via feeding blood vessels with or without surgery, topical application, e.g., in conjunction with a wound dressing after surgery, by injection, by means of a catheter, by means of a suppository, or by means of an implant, said implant being of a porous, non-porous, or gelatinous material. According to some preferred embodiments, administration can be by direct injection e.g., via a syringe, at the site of a tumor or neoplastic or pre-neoplastic tissue.

The compounds may also be administered by any convenient route, for example by infusion or bolus injection, by absorption through epithelial linings (e.g., oral mucosa, rectal and intestinal mucosa, etc.), and may be administered together with other therapeutically active agents. It is preferred that administration is localized, but it may be systemic. In addition, it may be desirable to introduce the pharmaceutical compositions of the invention into the central nervous system by any suitable route, including intraventricular and intrathecal injection; intraventricular injection may be facilitated by an intraventricular catheter, for example, attached to a reservoir. Pulmonary administration can also be employed, e.g., by use of an inhaler or nebulizer, and formulation with an aerosolizing agent.

A compound of the present invention can be delivered in an immediate release or in a controlled release system. In one embodiment, an infusion pump may be used to administer a compound of the invention, such as one that is used for delivering chemotherapy to specific organs or tumors (see Buchwald et al., 1980, Surgery 88: 507; Saudek et al., 1989, N. Engl. J. Med. 321: 574). In a preferred form, a compound of the invention is administered in combination with a biodegradable, biocompatible polymeric implant, which releases the compound over a controlled period of time at a selected site. Examples of preferred polymeric materials include polyanhydrides, polyorthoesters, polyglycolic acid, polylactic acid, polyethylene vinyl acetate, copolymers and blends thereof (See, Medical applications of controlled release, Langer and Wise (eds.), 1974, CRC Pres., Boca Raton, Fla.). In yet another embodiment, a controlled release system can be placed in proximity of the therapeutic target, thus requiring only a fraction of the systemic dose.

Furthermore, at times, the pharmaceutical compositions may be formulated for parenteral administration (subcutaneous, intravenous, intraarterial, transdermal, intraperitoneal or intramuscular injection) and may include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that include suspending agents, solubilizers, thickening agents, stabilizers, and preservatives. Oils such as petroleum, animal, vegetable, or synthetic oils and soaps such as fatty alkali metal, ammonium, and triethanolamine salts, and suitable detergents may also be used for parenteral administration. The above formulations may also be used for direct intra-tumoral injection. Further, in order to minimize or eliminate irritation at the site of injection, the compositions may contain one or more nonionic surfactants. Suitable surfactants include polyethylene sorbitan fatty acid esters, such as sorbitan monooleate and the high molecular weight adducts of ethylene oxide with a hydrophobic base, formed by the condensation of propylene oxide with propylene glycol.

The parenteral formulations can be presented in unit-dose or multi-dose sealed containers, such as ampoules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets of the kind previously described and known in the art.

Alternatively, the combinations of the present invention can be used in hemodialysis such as leukophoresis and other related methods, e.g., blood is drawn from the patient by a variety of methods such as dialysis through a column/hollow fiber membrane, cartridge etc, is treated with the oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts and the chemotherapeutic drug Ex-vivo, and returned to the patient following treatment. Such treatment methods are well known and described in the art. See, e.g., Kolho et al. (J. Med. Virol. 1993, 40(4): 318-21); Ting et at (Transplantation, 1978, 25(1): 31-3); the contents of which are hereby incorporated by reference in their entirety.

The present invention is also directed to the use of a dimeric or polymeric guanidine derivative as defined above for the preparation of a cytostatically active liposomal drug composition.

Further, the present invention is directed to the use of a dimeric or polymeric guanidine derivative as defined above for the preparation of an antimicrobial drug composition.

A still further aspect of the present invention is a process for therapeutically treating human beings and animals, characterized in that a drug composition according to the present invention as defined above is injected into a human being or an animal in need of that.

The liposomes were prepared according to the method, which is described in detail in EP 1 337 322 and U.S. Pat. No. 6,843,942.

The liposome preparation method can be described as modified ethanol injection system. Liposomes are produced by the crossflow injection technique which is a highly reproducible technology for the active and/or passive incorporation of a variety of pharmaceutical active substances into liposomes with defined size distribution. The production equipment is designed to meet several requirements such as simplicity, ruggedness and easy handling in sterilization procedures

In brief, the lipid components, in particular DMPC, DPPC, DMPG, DSPE-PEG-2000 and cholesterol, are dissolved in a water miscible organic solvent, especially ethanol. The polymeric guanidine derivative, preferably poly-[2-(2-ethoxyethoxyethyl)guanidium hydrochloride], is suspended in PBS or in physiological sodium chloride solution. The aqueous phases are either kept at 55° C. or at room temperature. The injection module, wherein the solvent and the aqueous phases are mixed, is equipped with an injection hole of 350 μm diameter. The lipid solution is merged with the aqueous active ingredient solution at an injection pressure of 5 bar and flow rate of the aqueous phase between 200-500 ml/min.

Liposome size and homogeneity can be controlled by the local lipid concentration at the injection/mixing point. The local lipid concentration is influenced by the lipid concentration in the organic solvent, the injection pressure, the injection bore diameter and the flow rate of the aqueous phase. Additional influence on the liposome size have the process temperature, the ionic strength of the aqueous phase and the osmolality of the chosen buffer system. Subsequent filtration steps are performed to remove untapped API and residual organic solvent.

Standard liposomes, consisting of phospholipids and cholesterol, can be prepared with the procedure described above. Typical formulations contain DMPC and cholesterol, DMPC, DMPG and cholesterol , DMPC, DPPC and cholesterol or pure DPPC. After pre-formulation of the liposomes with the above standard method stepwise downsizing of the liposomes can be accomplished by extrusion through straight pore polycarbonate filters. Liposomes formulated and downsized in the presence of polymeric guanidine derivates as described tend to form larger structures than in the absence of these substances.

Two different formulation, namely DMPC and cholesterol and DMPC, DMPG and cholesterol, were chosen for additional studies on the effect of polymeric guanidine derivatives on the size of the formed liposomes using standard lipid compositions and standard processes. For these studies, empty as well as poly-[2-(2-ethoxyethoxyethyl)guanidium hydrochloride] encapsulated liposomes were produced. A summary of these batches is given below.

In order to verify the hypothesis, that polymeric guanidine derivates interact with the liposomal surfaces, batch 1-4 were analysed with respect to the phase transition temperature of the liposomal membranes by differential scanning calorimetry (DSC). These experiments should clarify, if the API interacts with the head groups or with the backbones of the lipids. If a change in the phase transition occurs, API would be integrated within the membrane, otherwise just attached to the membrane surface.

The data of the DSC-scans shows clearly, that no difference in the main transition temperature can be observed between liposomes, which are prepared with or w/o API. Therefore it can be concluded, that there is no interaction between the polymeric guanidine derivates and the inner core of the liposomal membranes

Additional experiments were performed with the empty liposome in presence of free poly-[2-(2-ethoxyethoxyethyl)guanidium hydrochloride]. In these experiments, the empty liposome samples were spiked with poly-[2-(2-ethoxyethoxyethyl)guanidium hydrochloride] and after 1 h incubation, the samples were analysed with respect to liposome size, size distribution and zeta potential.

The addition of the positively charged free poly-[2-(2-ethoxyethoxyethyl)guanidium hydrochloride] charged the zeta potential of both liposome suspensions markedly. The suspension #1, which was composed of DMPC and cholesterol changed from a neutral/slightly negative surface potential to a positive, whereas the originally negatively surface potential of the suspension #2 was decreased by 6 m V to-3.13 mV. The addition of polyguanidine derivates also influences the hydrodynamic radius of the liposomes, which is measured by dynamic light scattering. The result for the particle size is given by the z-average mean and for the homogeneity by the polydispersity index. The ‘neutral’ suspension shows an increase in vesicle size by approx. 30 nm, which can be related to membrane surface attached polyguanidine derivates. The negatively charged suspension #4 is stronger influenced by the addition of free positively charged polyguanidines. The former homogeneously distributed liposomes turn into large aggregates, which can not be determined by the standard size measurements. This strong tendency to form aggregates can be explained by the interaction between the negatively surface charge of the liposomes and the positively charged polymeric API.

On the contrary according to the present invention liposomal formulations of polymeric guanidine derivates with PEGylated lipids do not form larger structures and/or aggregates

Among other applications the present invention should be used in oncology. For this purpose passive tumour targeting after prolonged circulation in the blood stream should be achieved. As published in scientific literature passive targeting can be accomplished by introducing PEG-chains in the formulation of the drug.

In formulation experiments a mixture consisting of DMPC and they pegylated lipid DSPE-PEG-2000 was investigated. Compared to the first approach, where polymeric guanidine derivates were encapsulated in liposomes composed of standard phospholipids, there is no influence pf the API on the formulation behaviour of PEG-liposomes. Liposomes prepared in presence and absence of polymeric guanidine derivates do not differ in size range and homogeneity

In addition, the API concentration has no influence on formulation behaviour and API concentration did not influence liposomes size and homogeneity.

The current invention is not limited to these three examples, but includes substances such as polymeric biguanides and other polymeric guanidine derivates too.

The activity of the liposomal formulations according to the present invention have been tested in-vitro in different concentrations and in several cell lines using an H-thymidine-test as described above. However, the drug formulation described in example 1 has shown improved tolerability also in-vivo models.

A tolerance study conducted in mic showed that in contrast to the free API the liposomal encapsulated formulation according to the present invention is tolerated at a daily intravenous dosing regime at a dose of 2.5 mg/kg body weight. On a weekly basis even 5 mg/kg body weight have well been tolerated. Whereas non-liposomal formulations of polymeric guanidine derivates induce necrosis e.g. in the tail vein, this is not the case with the PEGylated liposomal formulation according to the present invention, which is the proof that the active ingredient is not accumulated at the injection site, but systemically distributed by the blood stream.

Based on this intravenous tolerance study in mice, a clinical 2 case study with a half-breed dog suffering from hemangiosarcoma Stage III (T2N0M1) with multiple lung metastases was conducted. The terminal clinical state of the patient and the request of his holder allowed for a therapy with the provided drug-liposomal formulated poly-[2-(2-ethoxyethoxyethyl)guanidinium hydrochloride] at a veterinary university center-in line with present scientific knowledge and the therapeutic possibilities in this disease and therefore is equivalent to a compassionate treatment attempt according to the declaration of Helsinki in human medicine and is ethically justifiable.

The dog was treated three times—on day 1, 3 and 8—with a dose 5 mg/kg body weight diluted in physiological sodium chloride solution by intravenous infusion. The therapy was wall tolerated and the dog did not show clinical signs of side effects and the blood counts were not affected by the therapy. Two weeks after start of the therapy the radiological control showed a disease stabilization of the lung lesions compared to the baseline CT-examination. The observed effect was accompanied by an improved clinical state and situation. Another important fact is that the white and red blood cells did not show a significant decline like under cytostatic therapy.

Further infusions were given on a daily basis on days 14 to 17 with a dose 2mg/kg body weight. By then the dog had survived for more than 30 days despite an initial prognosis at treatment start of a few days. After the therapies the dog showed a good clinical condition and regained its normal activity.

The drug composition according to the present invention appears to be relatively well tolerated and induced a disease stabilization in this terminally ill dog suffering from a far progressed hemangiosarcoma with multiple lung lesions, little to no hematological and organ toxicity was observed

### ABBREVIATIONS

API; Active pharmaceutical ingredient (In this patent application a polymeric guanidine derivative encapsulated in liposomes according to the present invention)

DMPC; 1,2-dimyristoyl-sn-glycero-3-phosphocholine

DPPC; 1,2 -dipalmitoyl-sn-glycero-3-phosphocholine

DMPG; 1,2-dimyristoyl-sn-glycero-3-phospo-(1′ -rac-glycerol)

DSPE-PEG 2000; 1,2-distearoyl-sn-glycero-3-phosphoethanolamine-N-[amino(polyethylene glycol)-2000]

PBS; Phosphate buffered saline

PES; Polyethersulfone

Pdl; Polydispersity Index

PEG; Polyethylenglycol

1. A method for the treatment of cancer in a subject, said method comprising administering to said subject a therapeutically effective amount of a compound selected from the group consisting of oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts, wherein said therapeutically effective amount of said compound is about 15-25 mg per kg body weight per day and has a lethal effect on cells of said cancer, and wherein said cancer is selected from the group consisting of breast cancer, colon cancer, lung cancer, pancreatic cancer, ovarian cancer, glioblastoma and leukemia.

2. The method according to claim 1, wherein the compound is oligo(2-(2-ethoxy)ethoxy ethyl guanidinium chloride) polyetheramines, Triethyleneglycol diamine.

3. The method according to claim 1, wherein the compound is poly (hexamethylendiamine guanidiniumchloride) polyetheramines, Triethyleneglycol diamine.

4. A method according to claim 1, wherein said cancer is selected from the group consisting of breast cancer, colon cancer, lung cancer, pancreatic cancer, ovarian cancer, glioblastoma and leukemia.

5. The method of claim 1, wherein the combination therapy has a synergistic therapeutic effect.

6. The method of claim 1, wherein the cancer is selected from the group consisting of solid tumors and non-solid tumors.

7. The method of claim 1, wherein the compound is very effective if you use a liposomal drug composition.

8. Liposomal drug composition containing: a dimeric or polymeric guanidine derivative or a pharmaceutically, polyetheramines, triethyleneglycol diamine acceptable salt thereof as drug substance, and a lipid modified by polyethylene glycole (PEG) polyetheramines, triethyleneglycol diamine acceptable salt thereof as drug substance, and a lipid modified by polyethylene glycole (PEG).

9. A liposomal drug composition according to claim 7, characterized in that said lipid is a phospholipid and said PEG is PEG₅₀₀-PEG₅₀₀₀.

10. A liposomal drug composition according to claims 7 and 8, wherein said polymeric guanidine derivative is one, which guanidine derivative is based on a diamine containing oxyalkylene chains between two amino groups, with the guanidine derivative representing a product of polycondensation between a guanidine acid addition salt and a diamine containing polyoxyalkylene chains between two amino groups.

11. A liposomal drug composition according to claim 8, characterized in that, among the representatives of the family of polyoxyalkylene guanidine salts, there are such using triethylene glycol diamine (relative molecular mass; 148), polyoxypropylene diamine as well as polyoxyethylene diamine,

12. A liposomal drug composition according to any of claims 8 to 11, characterized in that poly-[2-(2-ethoxyethoxyethyl)guanidinium hydrochloride] comprising at least 3 guanidinium groups is contained as the drug substance.

13. A drug composition according to claim 7, characterized in that the average molecular mass of the drug substance ranges from 500 to 3,000.

14. The use of a dimeric or polymeric guanidine derivative as defined in claims 7 and 8-9 for the preparation of a cytostatically active liposomal drug composition.

15. The use of a dimeric or polymeric guanidine derivative as defined in claims 7 and 8-9 for the preparation of an antimicrobial drug composition.

16. A process for therapeutically treating human beings and animals, characterized in that a drug composition according to claim 1-6 is injected into a human being or an animal in need of that.

17. The method of claim 1, wherein the at least one additional anti-cancer agent is selected from the group consisting of a biological agent.

18. The method of claim 48, wherein the at least one additional anti-cancer agent is a PGPR, Plant Growth Promoting Rhizobacteria, said biological agent is an antibody selected from the group consisting of Acinetobacter, Achromobacter, Aereobacter, Agrobacterium, Alcaligenes, Artrobacter, Azospirillum, Serratia, Bacillus, Burkholderia, Enterobacter, Erwinia, Flavobacterium, Microccocus, Pseudomonas, Rhizobium ve Xanthomonas.

19. The method of claim 1, wherein the antioxidant is humic acid leached from leonardite ore and its sodium/potassium salts.

20. The method of claim 1, wherein amino acids are from a group of L-cysteine, and L-arginine.

21. The method of claim 1, wherein enzymes are from a group glutaminase, Arginine decarboxylase, histidine decarboxylase (Lactobacillus), and carboxypeptidase.

22. The method of claim 1, wherein herbal plants are Aniseed (Anisi fructus), Barbados Aloes (Aloe barbadensis), Bearberry leaf (Uvae ursi folium), Bilberry Fruit (Myrtilli fructus), Birch Leaf (Betulae folium), Black Cohosh (Cimicifugae rhizoma), Black Currant Leaf (Ribis nigri folium), Black Horehound (Ballotae nigrae herba), Bogbean leaf (Menyanthidis trifoliatae folium), Burdock Root (Arctii radix), Butcher's Broom (Rusci rhizome), Cape Aloes (Aloe capensis), Cascara (Rhamni purshianae cortex), Centaury (Centaurii herba), Clove oil (Caryophylli aetheroleum), Cola (Colae semen), Comfrey root (Symphyti radix), Couch Grass Rhizome (Graminis rhizoma), Elder flower (Sambuci flos), Feverfew (Tanaceti parthenii herba), Frangula Bark (Frangulae cortex), Gentian Root (Gentianae radix),Grindelia (Grindeliae herba), Hamamelis bark (Hamamelidis cortex), Hamamelis leaf (Hamamelidis folium), Hamamelis water (Hamamelidis aqua), Hydrastis rhizoma (Goldenseal rhizome), Ispaghula Husk (Plantaginis ovatae testa), Java Tea (Orthosiphonis folium), Lady's Mantle (Alchemillae herba), Linseed (Lini semen), Mallow Flower (Malvae flos), Meadowsweet (Filipendulae ulmariae herba), Melissa leaf (Melissae folium), Myrrh (Myrrha), Mullein flower (Verbasci flos), Nettle Root (Urticae radix), Pelargonium Root (Pelargonii radix), Psyllium Seed (Psylli semen), Restharrow Root (Ononidis radix), Rhatany Root (Ratanhiae radix), Ribwort Plantain leaf/herb (Plantaginis lanceolatae folium/herba), Sage Leaf, Trilobed (Salviae trilobae folium), Tormentil (Tormentillae rhizoma), White Horehound (Marrubii herbal), Wild Pansy (Violae herba cum flore), Wild Thyme (Serpylli herba), Willow Bark (Salicis cortex).

23. The method of claim 1, wherein natural products are polyphenols such as EGCG, resveratrol, curcumin, and genistein.

24. The method of claim 1, wherein it contains cephamine and dopamine.

25. The method of claim 1, wherein the at least one additional anti-cancer agent is known to be effective in treating said group of cancer.

26. The method of claim 1, wherein oligo(2-(2-ethoxy)ethoxy ethyl guanidinium chloride), poly(hexamethylendiamine guanidiniumchloride), polyetheramines, Triethyleneglycol diamine, enzymes, PGPR, amino acids, antioxidants like humic acids and some natural products like phytotherapeutic plant extracts are administered simultaneously, sequentially or concurrently.
